(19) United States
(12) Patent Application Publication
(10) Pub. No.: US 2008/0102487 A1
(43) Pub. Date: May 1, 2008

(54) Method and apparatus for non-invasive rapid fungal specie (mold) identification having hyperspectral imagery

(75) Inventors: Haibo Yao, Slidell, LA (US); Zuzana Hruska, Covington, LA (US); Kevin Dicrispino, Harahan, LA (US); Robert L. Brown, Prairieville, LA (US); Thomas E. Cleveland, Mandeville, LA (US)

Correspondence Address: CROWELL & MORING LLP;INTELLECTUAL PROPERTY GROUP, P.O. BOX 14300, WASHINGTON, DC, 20044-4300, US

(73) Assignees: Institute for Technology Development, Stennis Space Center, MS (US); USDA Southern Regional Research Center, Baton Rouge, LA (US)

(21) Appl. No.: 11/590,747

(22) Filed: Nov. 1, 2006

(51) Int. Cl. C40B 20/04; G06F 19/00; C12Q 1/04

(52) U.S. Cl. 435/34; 702/19; 506/4

(57) ABSTRACT

In a method and apparatus for identifying and distinguishing fungal species, a hyperspectral imaging scanner is used to acquire hyperspectral image data for radiation obtained from a sample area in which at least one unknown fungal species is present. A computer compares the acquired hyperspectral image data with spectral signature data stored in a digital library, which includes spectral signature data for each one of a group of known fungal species, and identifies the fungal species, based on the result of such comparison. The spectral signature data stored in the digital library take into account, for each fungal species, spectral variations that can occur due to at least one of environmental and temporal influences. The computer comparison includes a pixel-by-pixel analysis of the degree of difference between acquired hyperspectral image data and the spectral signature data, so that a spatial distribution of identified fungal species can be determined for a sample area.

The U.S. Government has a paid-up license in this invention and the right in limited circumstances to require the patent owner to license others on reasonable terms as provided for by the terms of Specific Cooperative Agreement No. 58-6435-3-121 awarded by the U.S. Department of Agriculture (“USDA”), Southern Regional Research, New Orleans, La.

### BACKGROUND OF THE INVENTION

The present invention relates to an optical system for identification of fungal species (such as mold) in cultured laboratory samples. Furthermore, the system can also be deployed outside the laboratory, where fungal infestation occurs, for fungal species identification. More particularly, the invention is useful for identification of mold species discovered in human habitation and environment.

Molds are organisms in the taxonomic kingdom of fungi that reproduce by making spores. There are perhaps 100 to 200 molds that can be continuously found indoors. Allergic reactions are the most common mold health problem from exposure, such as allergic rhinitis, dermatitis, asthma associated aggravation and hypersensitivity pneumonitis. Toxigenic molds such as Aspergillus, Fusarium, Penicillium, Chaetomium and Stachybotrys can release chemicals called mycotoxins during the metabolic cycle that can be “toxic” to humans.

Currently the most common methods available for mold identification involve culturing fungal samples, and then applying microscopic observation or using molecular based assays (analytical approach).

Various commercial mold identification services are available, which rely on microscopic observation. Some of these are full service; that is, they inspect, collect, analyze, and remediate. For this purpose, some such services send a certified mold inspector to check a structure for mold contamination utilizing moisture measurements, air testing methods, swab, and tape lift, followed by lab analysis, and a report that includes survey findings and recommendations for mitigation. Other services require the consumer to purchase a collection kit, which includes instructions for sampling air and/or surface contamination. The samples are then sent to the vendor for laboratory analysis, with a turn around time that is measured in weeks. Kits of this type may be priced from approximately $20.00-$200.00. (For an additional fee, some services will conduct an analysis and return a result on an expedited basis.)

Traditional laboratory identification methods for fungal and microbial identification require culturing samples and microscopic identification by a trained mycologist. The approach utilizes microscopic images to observe mold spores. Mold species can thus be identified through morphological descriptions of the mold spore by a mycologist. One recent development of this method is the Digital DIS-10 System from Digital Diagnostic Systems, LLC, which uses digital microscopy imaging for mold spore image acquisition. The image is sent back to the laboratory via the Internet, and the mycologist at the lab analyzes and identifies submitted samples based on digital image reference database of fungal and mold spores, with a 24 hour turn around time.

Fungal identification kits provide a more efficient and economical culturing method prior to microscopic observation and identification. (See, for example, U.S. Pat. No. 4,867,316). The kit is completely self contained, sterile, ready for use and disposable. The most suitable use for the kit is the clinical laboratory where the results may be readily interpreted by a mycology expert. Outside the laboratory the kit may be used to collect and incubate samples, but reading of the results would be delayed several hours or possibly days.

Traditional microscopic observation methods are expensive and relatively labor intensive. A full service approach will require no input from the user but may be priced accordingly. A less than full service approach requires training in sample collection, and may require, by lease or purchase, operation of a digital microscope, and in addition requires some expertise with computers and data transfer. A mycologist or similar technician must analyze the samples once they are received from the customer.

U.S. Pat. No. 4,874,695 is an example of an analytical based approach to fungal and microbial identification, which uses enzyme detection kits for the identification of yeasts and other specific microorganisms. The advantage of the enzyme rapid identification kit is that the method is based on calorimetric detection of characteristic enzymes, using chromogenic substrates produced by individual fungi or yeasts, and therefore, is self indicative. Unfortunately, the kit is limited to detecting yeasts and yeast like organisms and requires extensive culturing (48-72 hr) and incubation (2-6 hr) periods before observing the calorimetric results.

More recently, genetically-based, polymerase chain reaction (PCR) techniques have been established as useful tools for the identification of fungal and bacterial isolates. PCR is used to enzymatically amplify a short, well defined part of a DNA strand many times, in an exponential manner, without using a living organism. Because of this process, theoretically, only a very small sample is required to identify a genetic fingerprint. The PCR technique requires several basic components including a DNA template, a DNA sample containing the region of the DNA fragment to be amplified; two primers that determine the beginning and end of the amplification region; Taq polymerase, the enzyme which copies the region to be amplified; nucleotides, from which new DNA is built; a buffer to provide optimal chemical environment for the reaction and several pieces of relatively costly lab equipment. The process is well established and theoretically very precise and reliable, although time consuming and subject to human error. Related systems provide unique DNA sequences which may be used to make oligonucleotide primers for PCR based analysis for identification of fungal pathogens (U.S. Pat. No. 6,080,543), Internal Transcribed Spacer (ITS) DNA sequences from the ribosomal RNA region for different strains of fungal pathogens found in cereals including Septoria, Pseudocercosporella, Microdochium, Mycospaerella and Fusarium (U.S. Pat. No. 5,814,453), as well as nucleic acid probes and primers for detecting a host of disease causing fungi in humans and animals as well as food samples (U.S. Pat. No. 6,180,339 B1).

The PCR technique is widely used in clinical laboratories for viral and bacterial diagnosis because it is very sensitive, quantitative and relatively fast (within 24 hr). The drawbacks of the technique are that it is technically demanding, can be costly, poses a high risk of contamination, and requires rigid quality control at every step.

The deficiencies in the visual inspection based and current analytical based identification techniques have pointed to the need to develop automated or semi-automated systems for the identification of fungi. The system should provide a non-invasive approach to identify fungal species in a short period of time, preferably in real time.

U.S. Pat. No. 6,610,983 B2 is an example of such a non-invasive rapid detection technique, which utilizes electromagnetic radiation for the detection of fungi that grow in moist areas of a structure. The method includes exposing a structure to first electromagnetic radiation including at least one wavelength absorbed by a fungi to be detected. The method also includes sensing second electromagnetic radiation from the structure. The method then determines whether the fungi are present in the structure, based on the amplitude of sensed second radiation. The patent essentially describes methods for detecting the presence or absence of fungi, but does not provide a process for actual fungal identification.

Similar systems have been developed that utilize a technique in which a suspicious item is irradiated with light having a frequency (for example, UV, visible near-infrared, and short wave near-infrared) such that it causes the emission of fluorescent radiation upon striking the target. The fluorescent light from the target is then measured and compared with a threshold value. If the light thus gathered exceeds the threshold, the detection algorithm can generate a signal indicating the presence of a target. Such a system is disclosed, for example in U.S. Pat. No. 4,622,469 for detecting rotten albumen in broken raw eggs, and U.S. Pat. No. 6,512,236 B2 for viewing patterns of fluorescently stained DNA, protein or other biological materials.

Hyperspectral imaging systems that directly capture hyperspectral images through measuring target spectral reflectance are known. Such a system is disclosed, for example in U.S. Pat. No. 5,782,770 for non-invasive diagnosis of tissue for cancer and U.S. Pat. No. 6,992,775 for retinal image acquisitions.

### SUMMARY OF THE INVENTION

Accordingly, one object of the present invention is to use spectral information of mold species for mold identification.

Another object of the invention is to provide an automated or semi-automated process and apparatus for non-invasive identification of fungal species.

Another object of the invention is to provide a method for processing light reflected from a fungal colony, which method produces a signal that reliably indicates the exact fungal species, and an apparatus which implements such method.

Still another object of the invention is to provide a fungal identification system that can identify different fungi. Such a system includes light illumination sources, image capture devices, a database of reference fungi, processing methods, and identification algorithms.

A further object of the invention is to provide a fungal identification system that can be deployed in a controlled environment such as analytical labs to identify different fungi. The lab based system is for identifying well cultured mold samples one at a time. Such a system includes an operation computer, light illumination sources, image capture devices, a database of reference fungi, processing methods, and identification algorithms.

Yet another object of the invention is to provide a portable fungal identification system that can be used in real in-situ conditions to identify different fungi. In real situations, multiple molds could appear at the same sites which require the system to identify multiple fungi simultaneously. Such a system includes a portable operation computer, light illumination sources, image capture devices, a database of reference fungi, processing methods, and identification algorithms.

Finally, a still further object of the invention is to provide a non-invasive fungal identification system that can quickly and accurately identify different fungi.

The task is complicated by the fact that the targets for spectral identification according to the invention are living organisms. The situation therefore differs fundamentally from the use of spectral identification techniques to identify minerals, for example, because a known mineral is represented by a single reference spectrum. It can therefore be easily identified by its spectral signature. The reflectance signature for a living organism, on the other hand, is affected by many variables, such as nutrient supply, stress levels, days of growth, growth environment, background, etc. Moreover, another complicating factor is the proposition that, like many micro-organisms, molds tend to grow in colonies, and a contaminated area may include several different mold species, distributed throughout. Moreover, across the spatial extent of the contaminated area, the influencing factors (nutrient supply, etc.) may vary as well, so that the actual spectral signature of each mold that is present in the colony varies spatially.

The inventors' research has discovered that different mold species have different spectral reflectance features which, despite the above difficulties, can be used for mold identification. That is, by providing a detailed spectral library which includes spectral data that account for all of the variables that affect their reflectance characteristics, and by capturing spectral data for multiple image pixels in a contaminated area, it is possible to identify multiple mold species that may be present there. In addition to simply producing a label for an unknown spectrum, the invention can thus label multiple spectra (or image pixels) simultaneously because a hyperspectral image provides data with both high spectral and high spatial resolution. The invention uses such full spectral information to distinguish among for even very similar mold species. The invention and its real-time result also allow the user to collect an infinite amount of samples and requires no knowledge of fungal morphology.

Accordingly, the objects and advantages set forth above are achieved by the fungal species identification method and apparatus according to the invention, which provides techniques and devices for identification of mold species via hyperspectral imaging. The hyperspectral image is a three dimensional “image” in which one dimension contains spectral information and the other two dimensions contain the spatial information. The spectral data of the image can be analyzed on a pixel-by-pixel basis for the identification of fungal species and its spatial extent. Thus, the techniques are non-invasive and do not require introduction of agents typically required to facilitate interaction with illumination sources. The techniques also have minimum requirements in mold sample preparation and can generate identification results in a short period of time once the image is acquired.

Narrow band spectral reflectance across a wide spectral range (for example, UV, visible near-infrared, and short wave near-infrared) can provide rich spectral signature information regarding the suspicious targets. (Such spectral signature information exists separately for each pixel in a hyperspectral image.) The spectral signature can then be used for specific targets such as fungi identification. The identification process according to the invention involves irradiating fungi with electro-magnetic radiation (such as a light source working under certain wavelength range), measuring reflected radiation from the fungi with electronic devices (such as a CCD array that is sensitive in a certain wavelength range), composing a target signature from the captured signals, and implementing identification algorithms for fungal species identification. This invention applies this hyperspectral based approach and uses spectral information for fungal identification. Realization of the invention can be in the form of tabletop lab equipment.

The method according to the invention does not require any expert “hands on experience”, the analysis of results is automated, real time and objective. Furthermore, the “in situ” analysis eliminates the steps of shipment/transfer of samples and reduces the likelihood of human error in analyses or contamination by mishandling of samples.

Other objects, advantages and novel features of the present invention will become apparent from the following detailed description of the invention when considered in conjunction with the accompanying drawings.

### BRIEF DESCRIPTION OF THE DRAWINGS

FIG. 1 shows the spatial distribution and spectral signature of a mold colony;

FIG. 2 is a flow chart which shows an overview of the fungal species identification method according to the invention;

FIG. 3 is a schematic diagram of the fungal species identification apparatus according to the invention;

FIG. 4 is a graphic depiction of typical spectral signatures for several fungus species;

FIG. 5 is a schematic perspective view of a portable unit according to the invention, set up at a local infestation site; and

FIG. 6 is a flowchart for working system for simultaneous multiple mold identification.

### DETAILED DESCRIPTION OF THE INVENTION

The scanning and detection techniques utilized by the present invention are based on those disclosed in U.S. Pat. No. 6,166,373 (Mao), the disclosure of which is hereby incorporated by reference.

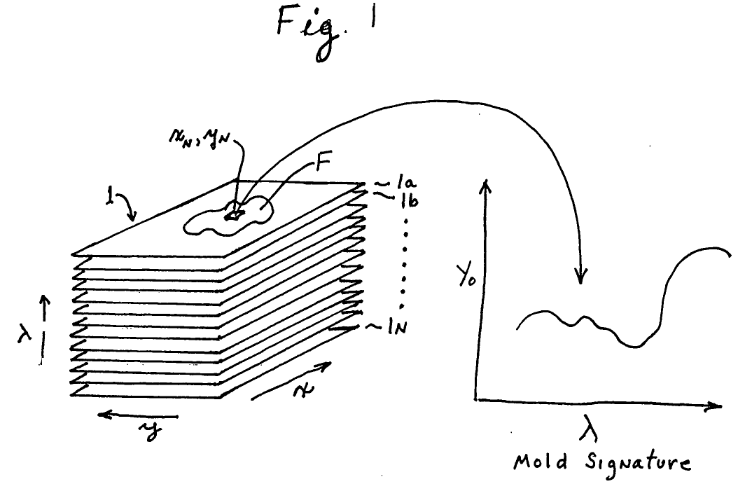

FIG. 1 herein illustrates a data set, sometimes referred to as a hyperspectral imaging cube 1, which results from the scanning of a sample using a hyperspectral scanning device. In such a data set, each x-y pane 1a-1n represents a spatial distribution of intensity values for an x-y matrix of individual pixels at a particular wavelength λ. That is, each of the “planes” 1a-1n can be thought of as an image of sensed radiation at a different wavelength. Collectively, the stack of such images forms a “cube”, in which spatial information is defined by the x and y axes, and spectral information is indicated along the λ axes, for each pixel in the x-y plane.

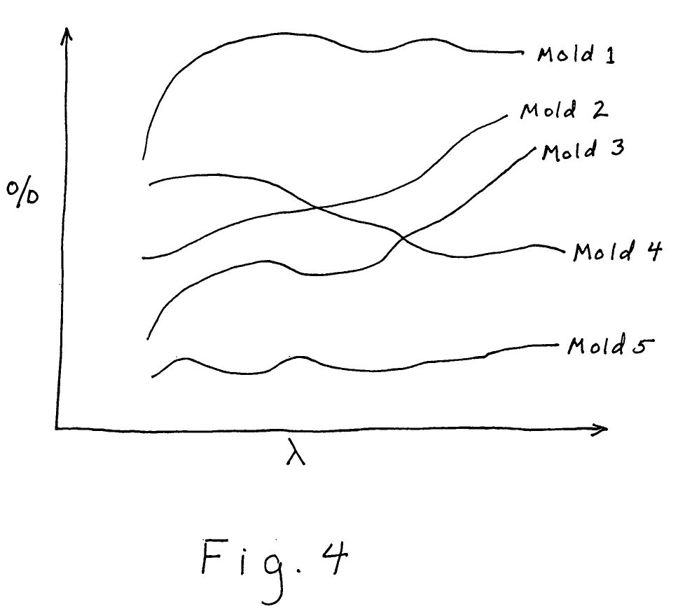

Thus, it can be seen that if a particular pixel is selected at a point (xₙ, yₙ) in the x-y plane that is located within a fungus sample F, the λ axis for that pixel will yield a so-called “spectral signature “that is uniquely associated with, and can be used to identify, the particular fungal specie that is present in that pixel. As depicted schematically in FIG. 4, each type of fungus or mold exhibits a unique spectral signature. The present invention, therefore, is based on the recognition of the fact that by using a hyperspectral scanner to acquire hyperspectral image data of an area that is suspected to harbor an unknown mold or fungus, it is possible not only to detect the presence or absence of a mold or fungus, as performed for example in U.S. Pat. No. 6,610,983, but also to identify the particular type or types of mold or fungus from among a broad range of fungal species, using known spectral signatures for each type of mold or fungus, previously determined from known samples.

The invention includes an imaging spectrometer for hyperspectral data acquisition, a digital mold reflectance library of all of the common molds that are found in environments where the system is to be used, and appropriate identification algorithm/software. The identification process is completely non-invasive, and rapid for real time work. The procedure can be automated. Its decision is objective because there is no human judgment involved.

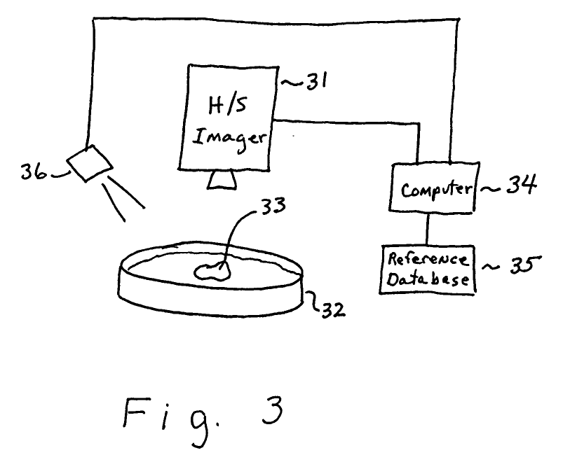

FIG. 3 is a simplified schematic illustration of a system for performing the fungus identification method according to the present invention. A hyperspectral imager (imaging spectrometer) 31 of the type disclosed for example in U.S. Pat. No. 6,166,373 (“Focal Plane Scanner with Reciprocating Spatial Window”) is arranged to scan a target, such as a Petri dish 32, which contains a sample of a mold 33 that is to be identified. The hyperspectral image data thus acquired by the imager 31 are input to a computer 34, which has stored therein image processing software which is capable of identifying the unknown mold sample by comparing its spectral signature with those contained in a reference database 35. Image processing software of this general type is known, and need not be described further herein. Optionally, although not mandatory, the system may also include a light source 36 which is controlled by the computer 34 for illuminating the same 33.

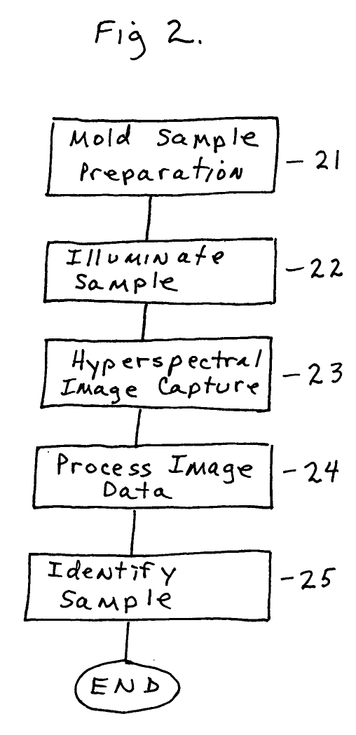

FIG. 2 is a flow diagram that illustrates the steps of an embodiment of the method according to the present invention. In a first step 21 a mold or fungus sample is prepared and positioned to be scanned by a hyperspectral scanner. It is then illuminated in step 22 (optional) and the scanner thereafter captures hyperspectral image data that include the mold or fungus sample in step 23.

In step 24, the computer 34 (FIG. 3) processes the hyperspectral image data to identify the particular mold or fungus in step 25. The image processing step includes reading in the captured hyperspectral image data, and comparing the spectral signature for the respective pixels within the mold or fungus sample with the stored reference signatures for various known types of molds or fungi contained in the reference database 35. Based on the result of such comparison, the computer 34 is able to identify the type or types of mold or fungus, automatically, objectively and without human intervention or analysis.

Spectral identification of the unknown mold can be implemented, for example, using common spectral matching algorithms such as binary encoding, Spectral Angle Mapper, and spectral feature fitting. A common feature of these spectral analysis algorithms is the calculation of a certain “distance” between each library spectrum and the unknown spectra. For example, Spectral Angle Mapper (SAM) matches unknown spectra to reference library spectra in n-dimensions using a physically-based spectral classification method. The algorithm regards the spectrum as vectors and compares the angle between each library spectrum and the unknown spectra in n-dimensional space. Smaller angles (or distance in general) represent closer matches to the library spectrum.

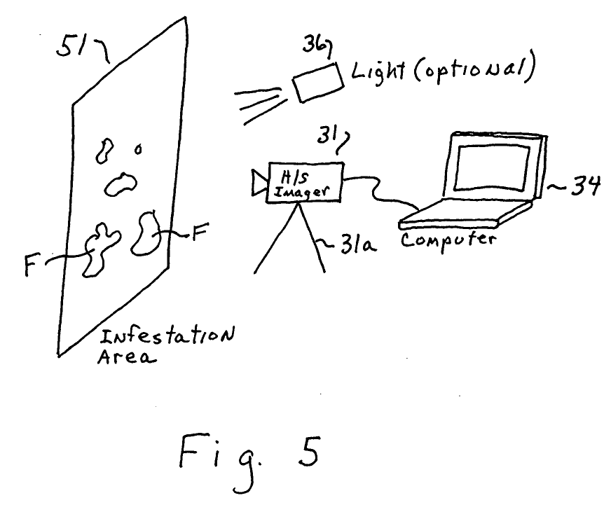

The method according to the invention can also be used to great advantage to analyze and identify mold infestations at remote locations, such as in flood zones, where molds and contaminations can pose serious health risks. Such a remote setup is depicted, for example, in FIG. 5, which shows a hyperspectral scanner 31 supported on a tripod 31a opposite an infestation area 51, which may be the wall of a building which has been submerged in flood water, and may harbor multiple types of fungi and molds. The imager 31 is coupled to the computer 34, which has stored therein imaging processing software and a reference database as discussed previously.

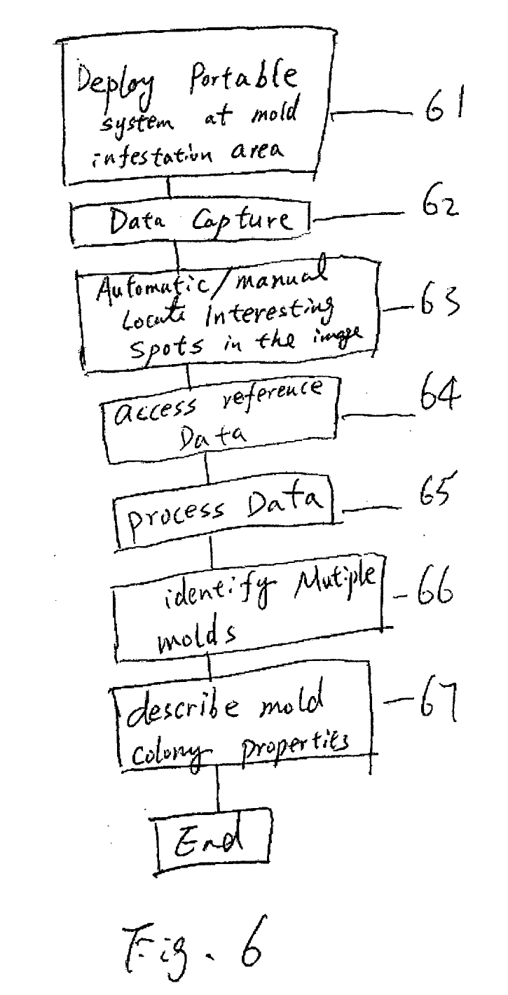

As shown in FIG. 6, when in use, the portable system is deployed at an infestation area in step 61, and hyperspectral image data from an infestation area are acquired by the scanner 31 in step 62. Within the image, the area of interest is located through either a known automatic process or manual process in step 63. The automatic process for locating an area of interest can be an unsupervised classification approach which is widely available commercially. The manual process can be an on screen digitizing process to select the region of interest. The computer then accesses the known spectral signature data stored in the reference database (step 64) and compares pixel-by-pixel the spectral signature data in the acquired hyperspectral imager in step 65. As a result of such comparisons, the computer is able to identify each of multiple types of molds or fungi which are present in the infestation area (step 66). In addition, step 67 can be used to describe properties of the mold colony, such as its size, possible inoculation point, fully developed and immature regions, and presence/absence of growth rings, etc.

The identification process according to the invention is completely non-invasive, and can be automated to achieve rapid results. Its decision is also objective, because no human analysis or judgment is involved.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A method for identifying and distinguishing fungal species, said method comprising:
using a hyperspectral imaging scanner to acquire hyperspectral image data for radiation obtained from a sample area in which at least one unknown fungal species is present;
providing a digital library which includes spectral signature data for each one of a group of known fungal species;
comparing said acquired hyperspectral image data with said spectral signature data contained in said digital library; and
identifying said fungal species based on a result of said comparing step.

2. The method according to claim 1, wherein:
said digital library includes spectral signature data which take into account, for each of said fungal species, spectral variations that can occur due to at least one of environmental and temporal influences;
said comparing step includes a pixel-by-pixel analysis of a degree of difference between acquired hyperspectral image data and said spectral signature data; and
said identifying step includes identifying a spatial distribution of at least one fungal species present in the sample area.

3. The method according to claim 2, wherein said identifying step includes:
distinguishing among a plurality of fungal species that are present in the sample area; and
determining a spatial distribution of each fungal species within the sample area.

4. The method according to claim 3, wherein said identifying step further comprises providing information regarding at least one of fungal colony size, growth of days, growth patterns and potential inoculation locations.

5. Apparatus for identifying and distinguishing fungal species present in a sample area, said apparatus comprising:
a hyperspectral image scanning device;
a computer readable memory having stored therein a digital library which includes spectral signature data for each one of a group of known fungal species; and
a computer having a memory encoded with a program for causing said computer to compare hyperspectral image data acquired by said scanning device with said spectral signature data contained in said digital library.

6. The apparatus according to claim 5, wherein:
said digital library includes spectral signature data which take into account, for each of said fungal species, spectral variations that can occur due to at least one of environmental and temporal influences; and
said program causes said computer to perform a pixel-by-pixel analysis of a degree of difference between acquired hyperspectral image data and said spectral signature data.

7. The apparatus according to claim 5, wherein said program causes said computer to:
distinguish among a plurality of fungal species that are present in the sample area; and
determine a spatial distribution of each fungal species within the sample area.

8. The apparatus according to claim 5, wherein:
said hyperspectral image scanning device is portable; and
said sample area comprises a contaminated remote location.

9. The apparatus according to claim 8, further comprising an illumination source for illuminating the sample area.

10. The apparatus according to claim 8, wherein:
said computer is situated at a site that is separated from the contaminated remote location; and
said scanning device is coupled in communication with said computer via a wide area computer network.

11. The apparatus according to claim 10, wherein said computer network comprises a globally distributed computer network.
